(19) United States
(12) Patent Application Publication
(10) Pub. No.: US 2025/0131560 A1
(43) Pub. Date: Apr. 24, 2025

(54) METHOD FOR ANALYZING CONDITION OF KNEE JOINT AND DEVICE FOR PERFORMING SAME

(71) Applicant: CRESCOM CO., LTD., Seongnam-si, Gyeonggi-do (KR)

(72) Inventor: Jae Joon LEE, Yongin-si (KR)

(73) Assignee: CRESCOM CO., LTD., Seongnam-si, Gyeonggi-do (KR)

(21) Appl. No.: 18/836,629

(22) PCT Filed: Aug. 18, 2022
(86) PCT No.: PCT/KR2022/012329

§ 371 Date: Aug. 7, 2024

(30) Foreign Application Priority Data
Feb. 8, 2022 (KR) 10-2022-0015946

(51) Int. Cl. G06T 7/00

(52) U.S. Cl. CPC G06T 7/0012; G06T 2207/20081; G06T 2207/20084; G06T 2207/30008

(57) ABSTRACT

A medical image analysis method according to an embodiment of the present application comprises the steps of: acquiring a medical image to be analyzed; acquiring a knee detection model that has been trained; acquiring a first feature region related to a first knee from the medical image through the knee detection model; acquiring a second feature region related to a second knee from the medical image through the knee detection model; and acquiring a first knee image to be analyzed, related to the first knee, and a second knee image to be analyzed, related to the second knee, on the basis of the first feature region and the second feature region of the medical image.

### TECHNICAL FIELD

The present application relates to a medical image analysis method, a medical image analysis apparatus, and a medical image analysis system. More particularly, the present application relates to a method of analyzing a condition of a knee joint, a medical image analysis method of performing the same, and a medical image analysis system.

### BACKGROUND ART

With improvements in image segmentation technology, it has become possible to calculate diagnostic auxiliary indicators related to various diseases by segmenting medical images, and the field of medical image analysis has recently been attracting attention. In particular, medical image analysis technology for providing joint condition information is being researched in various fields.

In analyzing joint conditions, one of the most important factors is a joint spacing, and the reduction in the joint spacing is known to have an important relationship with rheumatoid arthritis, degenerative arthritis, a wear status of cartilage, joint conditions in various parts of the body, and the like. In particular, many studies have demonstrated that a joint spacing value has a significant relationship with joint pain.

Conventionally, however, joint conditions are estimated based on an absolute value of the joint spacing. However, the absolute value of the joint spacing may vary significantly depending on external factors such as a gender, a race, and a body type. In addition, the absolute value of the joint spacing may cause an error depending on a system and program of an imaging device that captures an image. In this case, even if an error on a millimeter scale occurs, the change in the value is large, and it may lead to inaccurate results in finding a reduction rate of a joint spacing compared to normal, comparing with other patients, or monitoring prognosis. In other words, there is a limit to finding the joint condition based on the absolute value of the joint spacing. Accordingly, there is a need to develop a medical image analysis method, a medical image analysis apparatus and a medical image analysis system capable of obtaining objective joint condition information while minimizing an influence of external factors.

### DISCLOSURE

### Technical Problem

One problem to be solved by the present disclosure provides a medical image analysis method, a medical image analysis apparatus, and a medical image analysis system for calculating joint condition information.

Problems to be solved by the present disclosure are not limited to the above-described objects, and objects that are not mentioned will be clearly understood by those skilled in the art to which the present disclosure pertains from the present specification and the accompanying drawings.

### Technical Solution

According to an embodiment of the present application, a medical image analysis method may include: acquiring a target medical image; acquiring a trained knee detection model; acquiring a first feature region related to a first knee from the target medical image through the knee detection model; acquiring a second feature region related to a second knee from the medical image through the knee detection model; and acquiring a first target knee image related to the first knee and a second target knee image related to the second knee based on the first feature region and the second feature region of the target medical image.

According to an embodiment of the present application, a medical image analysis apparatus includes: an image acquisition unit that acquires a target medical image; and a controller that acquires a target knee image to be analyzed based on the target medical image, in which the controller may be configured to acquire the target medical image, acquire a trained knee detection model, acquire a first feature region related to a first knee from the target medical image through the knee detection model, acquire a second feature region related to a second knee from the medical image through the knee detection model, and acquire a first target knee image related to the first knee and a second target knee image related to the second knee based on the first feature region and the second feature region of the target medical image.

Technical solutions of the present disclosure are not limited to the above-described solutions, and solutions that are not mentioned will be clearly understood by those skilled in the art to which the present disclosure pertains from the present specification and the accompanying drawings.

### Advantageous Effects

According to a medical image analysis method, a medical image analysis apparatus, and a medical image analysis system according to an embodiment of the present application, it is possible to acquire objective joint condition information by minimizing the influence of external factors such as a body type, a race, and a gender.

Effects of the present disclosure are not limited to the above-described effects, and effects that are not mentioned will be clearly understood by those skilled in the art to which the present disclosure pertains from the present specification and the accompanying drawings.

### DESCRIPTION OF DRAWINGS

FIG. 1 is a schematic diagram illustrating a medical image analysis system according to an embodiment of the present application.

FIG. 2 is a diagram illustrating operations of the medical image analysis apparatus according to the embodiment of the present application.

FIG. 3 is a flowchart illustrating a method of analyzing a medical image according to an embodiment of the present application.

FIG. 4 is a flowchart embodying an operation of acquiring a target knee image according to an embodiment of the present application.

FIG. 5 is a diagram illustrating an aspect of acquiring first and second target knee images according to an embodiment of the present application.

FIG. 6 is a diagram illustrating a method of training a knee detection model according to an embodiment of the present application.

FIG. 7 is a flowchart embodying an operation of detecting a target joint spacing region according to an embodiment of the present application.

FIG. 8 is a diagram illustrating an aspect of detecting a region of interest (ROI) and a target joint spacing region according to an embodiment of the present application.

FIG. 9 is a flowchart illustrating a method of training a neural network model for acquiring a target joint spacing region according to an embodiment of the present application.

FIG. 10 is a flowchart illustrating an aspect of training a neural network model for acquiring a target joint spacing region according to an embodiment of the present application.

FIG. 11 is a flowchart embodying an operation of detecting a target joint spacing region according to an embodiment of the present application.

FIG. 12 is a schematic diagram illustrating an aspect of acquiring a target joint spacing region using a trained neural network model according to an embodiment of the present application.

FIG. 13 is a flowchart embodying an operation of acquiring a first value related to a width of a joint part according to an embodiment of the present application.

FIG. 14 is a diagram illustrating an aspect of detecting a first point and a second point according to an embodiment of the present application.

FIG. 15 is a diagram illustrating an aspect of acquiring a first value according to an embodiment of the present application.

FIG. 16 is a flowchart embodying an operation of acquiring a second value related to the joint spacing according to an embodiment of the present application.

FIG. 17 is a diagram illustrating an aspect of acquiring a second value according to an embodiment of the present application.

FIG. 18 is a diagram illustrating an example of visualization information of an analysis result of a medical image analysis apparatus according to an embodiment of the present application.

### BEST MODE

A medical image analysis method according to an embodiment of the present application may include: acquiring a target medical image; acquiring a trained knee detection model; acquiring a first feature region related to a first knee from the target medical image through the knee detection model; acquiring a second feature region related to a second knee from the medical image through the knee detection model; and acquiring a first target knee image related to the first knee and a second target knee image related to the second knee based on the first feature region and the second feature region of the target medical image.

According to an embodiment of the present application, the acquiring of the first target knee image related to the first knee and the second target knee image related to the second knee may include: acquiring a first coordinate related to the first feature region; acquiring a second coordinate related to the second feature region; acquiring a reference coordinate by assigning a preset weight to each of the first and second coordinates; and dividing the target medical image into the first target knee image and the second target knee image based on the reference coordinate.

According to an embodiment of the present application, the first feature region may be a region of at least one of right femur medial, right fibula, left femur medial, left fibula, right letter, and left letter, and the second feature region may be a region of at least one of the right femur medial, the right fibula, the left femur medial, the left fibula, the right letter, and the left letter.

According to an embodiment of the present application, the knee detection model may be configured to receive the target medical image and detect the first feature region or the second feature region included in the target medical image.

According to an embodiment of the present application, the knee detection model may include an input layer that receives the medical image, an output layer that outputs a predicted value, and a hidden layer that connects the input layer and the output layer, may be trained based on a training set composed of the medical image and label information allocated to the feature region of the medical image, and may be trained by adjusting a parameter of a node included in the hidden layer based on a difference between the predicted value output through the output layer and the label information.

According to an embodiment of the present application, the label information may include a value defining at least one of right femur medial, right fibula, left femur medial, left fibula, right letter, and left letter.

According to an embodiment of the present application, a computer-readable recording medium on which a program for executing the medical image analysis method is recorded may be provided.

A medical image analysis apparatus according to an embodiment of the present application includes: an image acquisition unit that acquires a target medical image; and a controller that acquires a target knee image to be analyzed based on the target medical image, in which the controller may be configured to acquire the target medical image, acquire a trained knee detection model, acquire a first feature region related to a first knee from the target medical image through the knee detection model, acquire a second feature region related to a second knee from the medical image through the knee detection model, and acquire a first target knee image related to the first knee and a second target knee image related to the second knee based on the first feature region and the second feature region of the target medical image.

### Best Mode

Objects, features, and advantages of the present application will become more obvious from the following detailed description provided in relation to the accompanying drawings. However, the present application may be variously modified and have several exemplary embodiments. Hereinafter, specific exemplary embodiments will be illustrated in the accompanying drawings and described in detail.

The same reference numerals denote the same constituent elements throughout the specification. Further, elements having the same function within the scope of the same idea illustrated in the drawings of each embodiment will be described using the same reference numerals, and overlapping description thereof will be omitted.

When it is determined that detailed description of known functions or configurations related to the present application may obscure the gist of the present application, detailed description thereof will be omitted. In addition, numbers (for example, “first,” “second,” etc.) used in the process of describing the present specification are only identification symbols for distinguishing one component from other components.

In addition, the terms “module” and “unit” for components used in the following embodiments are used only in order to easily make the disclosure. Therefore, these terms do not have meanings or roles that distinguish them from each other in themselves.

Singular forms are intended to include plural forms unless the context clearly indicates otherwise.

In the following embodiments, the terms “include,” “have,” and the like mean that a feature or element described in the specification is present, and do not preclude in advance the possibility that one or more other features or components may be added.

Sizes of components may be exaggerated or reduced in the accompanying drawings for convenience of explanation. For example, the size and thickness of each component illustrated in the drawings are randomly indicated for convenience of description, and the present disclosure is not necessarily limited to those that are illustrated those.

In a case where certain embodiments can be otherwise implemented, the order of specific processes may be performed different from the order in which the processes are described. For example, two processes described in succession may be performed substantially simultaneously, or may be performed in an order opposite to the order described.

In the following embodiments, when components are connected, it includes not only a case where components are directly connected but also a case where components are indirectly connected via certain component interposed between the components.

For example, in the present specification, when components and the like are electrically connected, it includes not only a case where components are directly electrically connected, but also a case where components are indirectly electrically connected via certain component interposed between the components.

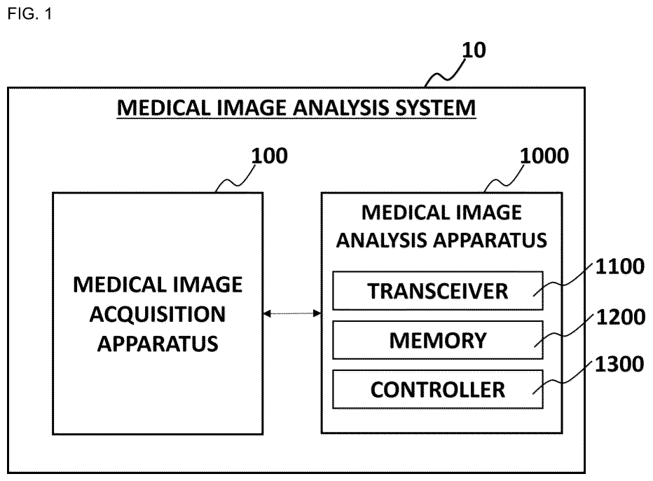

Hereinafter, a medical image analysis method, a medical image analysis apparatus, and a medical image analysis system of the present application will be described with reference to FIGS. 1 to 18.

FIG. 1 is a schematic diagram illustrating a medical image analysis system 10 according to an embodiment of the present application.

The medical image analysis system 10 according to an embodiment of the present application may include a medical image acquisition apparatus 100 and a medical image analysis apparatus 1000.

The medical image acquisition apparatus 100 may capture a medical image. For example, the medical image acquisition apparatus 100 may include any type of medical images, including magnetic resonance imaging, computerized tomography equipment, X-ray equipment, and the like. The medical image acquired by the medical image acquisition apparatus 100 may be a two-dimensional image. In this case, the medical image may include pixel information related to coordinates, color, intensity, and the like of a pixel. The medical image acquired by the medical image acquisition apparatus 100 may be a three-dimensional image. In this case, the medical image may include pixel information related to coordinates, color, intensity, and the like of a voxel.

The medical image analysis apparatus 1000 according to an embodiment of the present application may acquire joint condition information by analyzing a medical image. More specifically, the medical image analysis apparatus 1000 may detect a joint region from the medical image and quantify information related to a joint condition based on the joint region. Here, the joint region may include an inter-joint region (or joint spacing region), a bone region adjacent to the inter-joint region, and the like.

The medical image analysis apparatus 1000 according to the embodiment of the present application may include a transceiver 1100, a memory 1200, and a controller 1300.

The transceiver 1100 of the medical image analysis apparatus 1000 may communicate with any external device including the medical image acquisition apparatus 100. For example, the medical image analysis apparatus 1000 may receive a medical image captured by the medical image acquisition apparatus 100 through the transceiver 1100. Also, the medical image analysis apparatus 1000 may transmit the acquired joint condition information to any external device including the medical image acquisition apparatus 100 through the transceiver 1100.

The medical image analysis apparatus 1000 may be connected to a network through the transceiver to transmit/receive various types of data. The transceiver may largely include a wired type and a wireless type. Since the wired type and the wireless type have their respective strengths and weaknesses, in some cases, the wired type and the wireless type may be simultaneously provided in the medical image analysis apparatus 1000. Here, in the case of the wireless type, a wireless local area network (WLAN)-based communication method such as Wi-Fi may be mainly used. Alternatively, in the case of the wireless type, cellular communication, for example, Long Term Evolution (LTE) and a 5G-based communication method, may be used. However, the wireless communication protocol is not limited to the above-described example, and any appropriate wireless type communication method may be used. In the case of the wired type, local area network (LAN) or Universal Serial Bus (USB) communication is a representative example, and other methods are also possible.

The memory 1200 of the medical image analysis apparatus 1000 may store various types of information. Various types of data may be temporarily or semi-permanently stored in the memory 1200. An example of the memory 1200 may include a hard disk drive (HDD), a solid state drive (SSD), a flash memory, a read-only memory (ROM), a random access memory (RAM), or the like. The memory 1200 may be provided in a form in which it is embedded in the medical image analysis apparatus 1000 or in a detachable form. The memory 1200 may store various types of data necessary for the operation of the medical image analysis apparatus 1000 in addition to an operating system (OS) for driving the medical image analysis apparatus 1000 or a program for operating each component of the medical image analysis apparatus 1000.

The controller 1300 may control the overall operation of the medical image analysis apparatus 1000. For example, the controller 1300 may control the overall operation of the medical image analysis apparatus 1000 such as an operation of detecting a knee position from a target medical image and acquiring a target knee image, an operation of detecting a region of interest (ROI) or a target joint spacing region from the target medical image, and an operation of quantifying a joint spacing. Specifically, the controller 1300 may load and execute a program for the overall operation of the medical image analysis apparatus 1000 from the memory 1200. The processor may be implemented as an application processor (AP), a central processing unit (CPU), a microcontroller unit (MCU), or similar devices thereto according to hardware, software, or a combination thereof. In this case, the processor may be provided in an electronic circuit form processing an electrical signal to perform a control function in terms of hardware, and may be provided in a program or code form driving the hardware circuit in terms of software.

Meanwhile, although not illustrated in FIG. 1, the medical image analysis apparatus 1000 may include any appropriate input unit and/or output unit. Specifically, the medical image analysis apparatus 1000 may receive a user input necessary for analyzing the medical image through the input unit. For example, the medical image analysis apparatus 1000 may acquire a user input for allocating label information to each of the plurality of regions included in the medical image through the input unit. As another example, the medical image analysis apparatus 1000 may acquire, through the input unit, a user input for setting a section of interest of a joint spacing region for acquiring a joint spacing value.

Also, the medical image analysis apparatus 1000 may output, through an output unit, any analysis results including a result of comparing a target joint condition indicator and/or a target joint condition indicator to be described below with a reference joint condition indicator, etc.

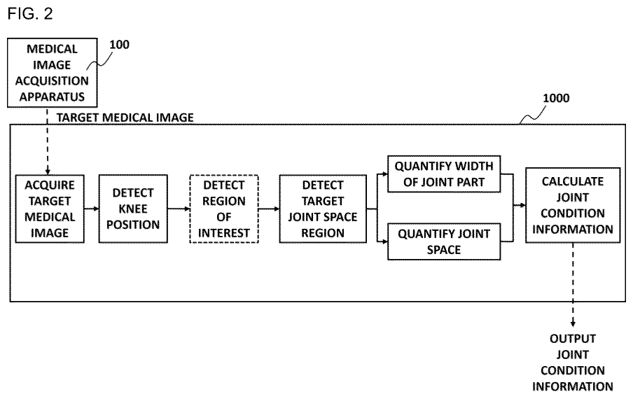

Hereinafter, an operation of the medical image analysis apparatus 1000 according to the embodiment of the present application will be described in detail with reference to FIGS. 2 to 18.

The medical image analysis apparatus 1000 according to an embodiment of the present application may detect the joint spacing region from the medical image. Also, the medical image analysis apparatus 1000 may calculate the joint condition information based on the joint spacing region.

FIG. 2 is a diagram illustrating operations of the medical image analysis apparatus 1000 according to the embodiment of the present application.

The medical image analysis apparatus 1000 according to the embodiment of the present application may include an image acquisition unit. Specifically, the image acquisition unit of the medical image analysis apparatus 1000 may acquire the target medical image acquired from the medical image acquisition apparatus 100. For example, the image acquisition unit may acquire the target medical image from any external device including the medical image acquisition apparatus 100 through the transceiver 1100.

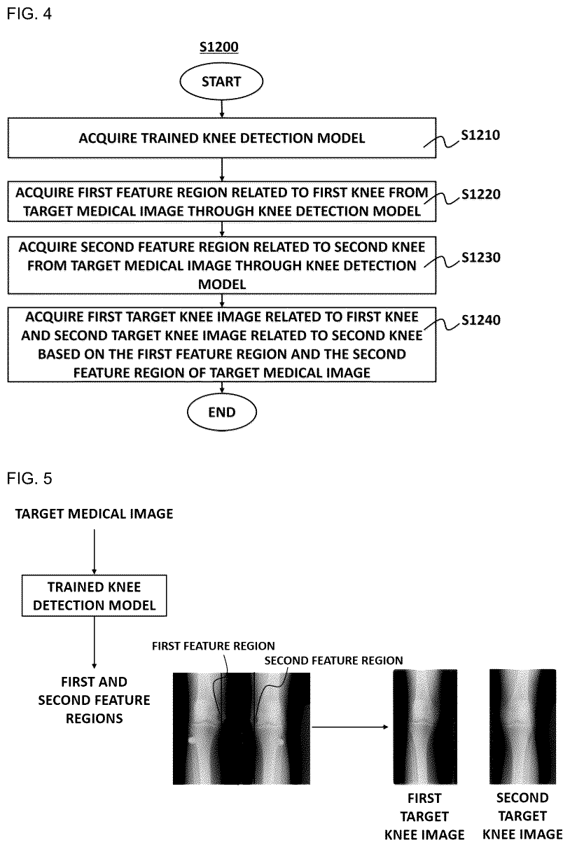

The medical image analysis apparatus 1000 according to the embodiment of the present application may include a knee position detector. Specifically, the knee position detector of the medical image analysis apparatus 1000 may detect feature regions related to the knee included in the target medical image and acquire a target knee image from the target medical image based on the feature regions. For example, the knee position detector may acquire a first feature region (e.g., a region of at least one of right femur medial, right fibula, left femur medial, left fibula, right letter, and left letter) related to a first knee, and a second feature region (e.g., a region of at least one of right femur medial, right fibula, left femur medial, left fibula, right letter, and left letter) related to a second knee from the target medical image through the trained knee detection model. In addition, the knee position detector may acquire a first target knee image related to the first knee and a second target knee image related to the second knee based on the first feature region and the second feature region. For example, the knee position detector may acquire a first coordinate corresponding to the first feature region and a second coordinate corresponding to the second feature region, and calculate a reference coordinate based on the first coordinates and the second coordinates. In this case, the knee position detector may be implemented to divide the target medical image into the first target knee image and the second target knee image based on the calculated reference coordinate. The content of acquiring the first target knee image and the second target knee image will be described in more detail in FIGS. 4 to 6.

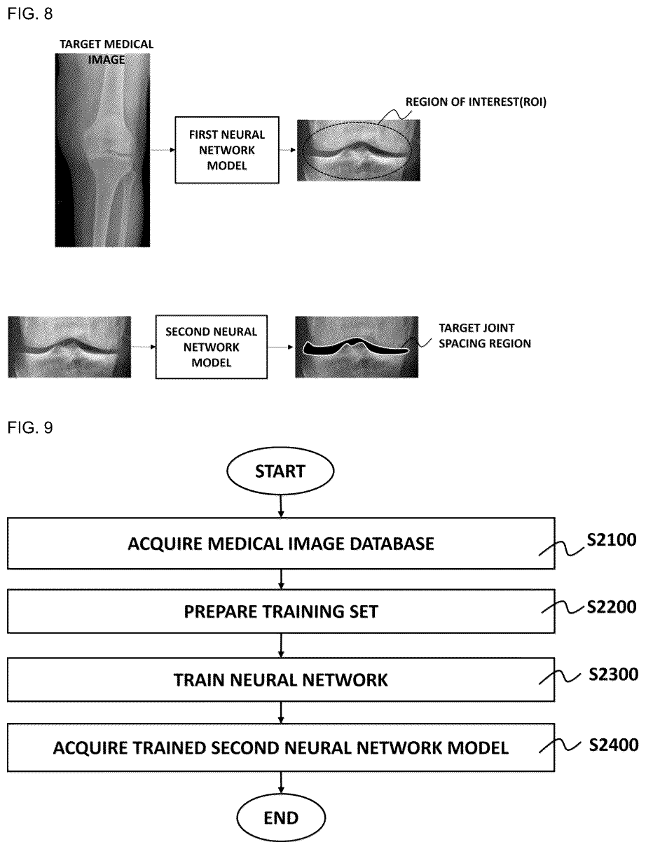

The medical image analysis apparatus 1000 according to the embodiment of the present application may include an ROI detector. The ROI detector of the medical image analysis apparatus 1000 according to an embodiment of the present application may detect the ROI included in the target medical image. For example, the ROI detector may detect an ROI including a joint region from the target medical image. Here, the joint region may include the joint spacing region and/or the bone region adjacent to the joint spacing region as described above. As another example, the ROI detector may detect a knee region, a cartilage region, and/or a main occurrence region (e.g., a region related to medial femur, femur lateral, tibia medial, tibia lateral, etc.) of osteophyte from the target medical image. Specifically, the ROI detector may detect the ROI using an artificial intelligence technique. The operation of detecting the ROI will be described in detail with reference to FIGS. 7 and 8. However, the operation of detecting the ROI from the target medical image may be omitted. For example, when the joint spacing region, which will be described later, can be directly detected from the target medical image, the operation of detecting the ROI in the ROI detector may be omitted.

The medical image analysis apparatus 1000 according to the embodiment of the present application may include a joint spacing region detector. The joint spacing region detector of the medical image analysis apparatus 1000 according to an embodiment of the present application may detect the target joint spacing region from the target medical image. As an example, the joint spacing region detector may detect the target joint spacing region from the ROI including the joint region included in the target medical image. Here, the target joint spacing region may refer to any inter-joint region included in the target medical image. According to the embodiment, the joint spacing region detector may acquire the target joint spacing region by using the artificial intelligence technique. For example, the joint spacing region detector may acquire the target joint spacing region using the neural network model trained based on the training set in which the inter-joint region (or joint spacing region) is allocated (or labeled) in the medical image. The content of acquiring the target joint spacing region will be described in detail with reference to FIGS. 7 to 12.

The medical image analysis apparatus 1000 according to the embodiment of the present application may include a joint condition quantification analyzer. In this case, the joint condition quantization analyzer may include a joint width analyzer and a joint spacing analyzer. The joint condition quantization analyzer of the medical image analysis apparatus 1000 according to the embodiment of the present application may calculate the joint condition information based on the joint region and/or the target joint spacing region.

The joint width analyzer of the medical image analysis apparatus 1000 may perform an operation of quantifying the width of the joint part based on the joint region included in the target medical image. For example, the joint width analyzer of the medical image analysis apparatus 1000 may detect an outer point of the joint part by using any image processing technique or artificial intelligence technique, and may calculate quantification information related to the width of the joint part based on coordinate information of the outer point. The content of quantifying the width of the joint part will be described in detail with reference to FIGS. 13 to 15.

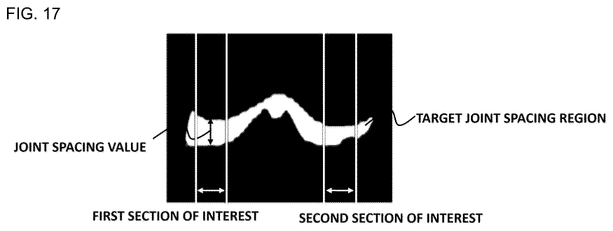

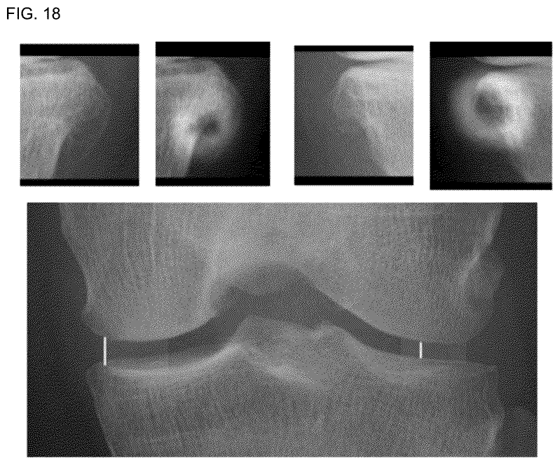

The joint spacing analyzer of the medical image analysis apparatus 1000 according to the embodiment of the present application may perform an operation of quantifying the joint spacing based on the target joint spacing region. For example, the joint spacing analyzer of the medical image analysis apparatus 1000 may acquire a section of interest related to the target joint spacing region and acquire at least one joint spacing value within the section of interest. Also, the joint spacing analyzer of the medical image analysis apparatus 1000 may calculate the quantification information related to the joint spacing based on at least one joint spacing value. The content of quantifying the joint spacing will be described in detail with reference to FIGS. 16 and 17.

The medical image analysis apparatus 1000 according to the embodiment of the present application may calculate the joint condition information based on the quantitative information related to the width of the joint part and the quantitative information related to the joint spacing. For example, the joint condition quantification analyzer of the medical image analysis apparatus 1000 may calculate a joint condition indicator indicating a joint condition based on the quantitative information related to the width of the joint part and the quantitative information related to the joint spacing.

Meanwhile, although not illustrated in FIG. 2, the medical image analysis apparatus 1000 according to an embodiment of the present application may include a medical image preprocessor. Specifically, the medical image preprocessor of the medical image analysis apparatus 1000 may perform preprocessing on any medical image used for medical image analysis, including the target medical image and/or the first and second target knee images to be described below. For example, a medical image preprocessor may perform preprocessing, such as contrast limited adaptive histogram equalization (CLAHE), histogram matching, and/or normalization, on medical images. As another example, the medical image preprocessor may perform preprocessing on a first medical image with a first image quality, perform preprocessing on a second medical image with a second image quality, and apply an ensemble technique to the preprocessed first medical image and second medical image. The ensemble technique is an algorithm that finds an optimal solution by averaging multiple single models or combining the results of different models to improve the performance of the existing models. The medical image preprocessor according to an embodiment of the present application may provide an effect of acquiring an average of result values of images obtained by performing preprocessing on a plurality of medical images with different image quality using the ensemble technique and deriving stable image analysis results for various image qualities based on the acquired average. As another example, the medical image preprocessor may perform preprocessing for data enhancement, such as flipping, rotation, and/or increase or decrease in noise on medical images. For example, the first target knee image, which will be described below, may be related to the left knee, and the second target knee image, which will be described below, may be related to the right knee. In this case, any neural network models used for image analysis may have been trained through images related to a specific knee. In this case, the medical image preprocessor can increase the accuracy of joint condition analysis through the preprocessing that flips or rotates the left knee image to the right knee image.

However, the above-described preprocessing technique is only an example, and any appropriate image processing technique may be used to increase the accuracy of the joint condition analysis. Meanwhile, the target medical image and/or the first and second target knee images referred to below may be interpreted with meanings encompassing the preprocessed images.

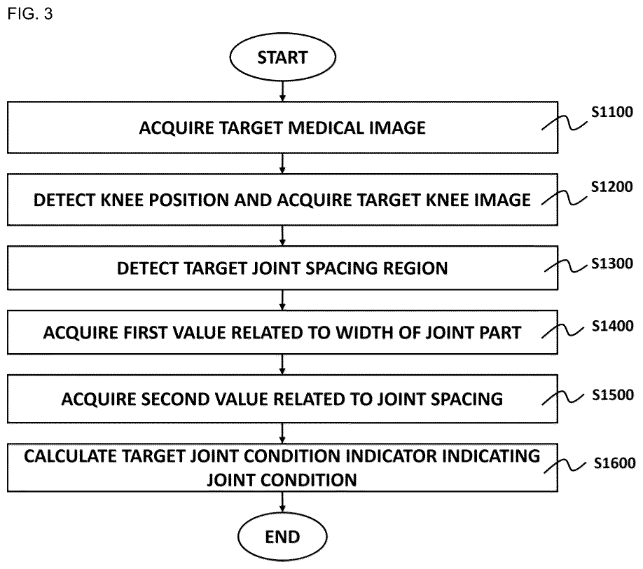

Referring to FIG. 3, FIG. 3 is a flowchart illustrating a method of analyzing a medical image according to an embodiment of the present application.

The medical image analysis method according to an embodiment of the present application may include acquiring the target medical image (S1100), detecting the knee position and acquiring the target knee image (S1200), detecting the target joint spacing region (S1300), acquiring a first value related to the width of the joint part (S1400), acquiring a second value related to joint spacing (S1500), and calculating a target joint condition indicator indicating the joint condition (S1600).

In the acquiring of the target medical image (S1100), the medical image analysis apparatus 1000 may acquire the target medical image to be analyzed. For example, the medical image analysis apparatus 1000 may acquire the target medical image from the medical image acquisition apparatus 100 or any external device including a database through the transceiver 1100.

In the detecting of the knee position and acquiring of the target knee image (S1200), the medical image analysis apparatus 1000 may detect feature regions related to a knee included in the target medical image and acquire the target medical image to be analyzed from the target medical image based on the feature regions.

Referring to FIGS. 4 and 5, FIG. 4 is a flowchart embodying an operation of acquiring a target knee image according to an embodiment of the present application. FIG. 5 is a diagram illustrating an aspect of acquiring first and second target knee images according to an embodiment of the present application.

The detecting of the knee position and acquiring of the target knee image according to an embodiment of the present application may include acquiring the trained knee detection model (S1210) acquiring the first feature region related to the first knee from the target medical image through the knee detection model (S1220), acquiring the second feature region related to the second knee from the target medical image through the knee detection model (S1230), and acquiring the first target knee image related to the first knee and the second target knee image related to the second knee based on the first feature region and the second feature region of the target medical image (S1240).

In the acquiring of the trained knee detection model (S1210), the medical image analysis apparatus 1000 may acquire the trained knee detection model. Specifically, the medical image analysis apparatus 1000 may acquire any execution data (e.g., layer information, operation information, and/or parameters of the knee detection model) for executing the trained knee detection model.

In the acquiring of the first feature region related to the first knee from the target medical image through the knee detection model (S1220), the medical image analysis apparatus 1000 may acquire a first feature region (e.g., a region of at least one of right femur medial, right fibula, left femur medial, left fibula, right letter, and left letter) related to a first knee (e.g., the left knee) of a patient from the target medical image through the knee detection model. Specifically, the knee detection model may be configured to receive the target medical image and output the first feature region of the target medical image. The medical image analysis apparatus 1000 may acquire the first feature region related to the first knee output through the output layer of the knee detection model.

In the acquiring of the first feature region related to the first knee from the target medical image through the knee detection model (S1220), the medical image analysis apparatus 1000 may acquire a first feature region (e.g., a region of at least one of right femur medial, right fibula, left femur medial, left fibula, right letter, and left letter) related to a first knee (e.g., the left knee) of a patient from the target medical image through the knee detection model. Specifically, the knee detection model may be configured to receive the target medical image and output the second feature region of the target medical image. The medical image analysis apparatus 1000 may acquire the second feature region related to the second knee output through the output layer of the knee detection model.

In the acquiring of the first target knee image related to the first knee and the second target knee image related to the second knee based on the first feature region and the second feature region of the target medical image (S1240), the medical image analysis apparatus 1000 may acquire the first target knee image and the second target knee image based on the first feature region related to the first knee and the second feature region related to the second knee included in the target knee image. Specifically, the medical image analysis apparatus 1000 may acquire the first coordinate corresponding to the first feature region related to the first knee included in the target knee image and the second coordinate corresponding to the second feature region related to the second knee included in the target knee image, and divide the target medical image into the first target knee image and the second target knee image based on the first and second coordinates.

As an example, the medical image analysis apparatus 1000 may acquire the reference coordinate and/or a reference line passing through the reference coordinate by weighting the first coordinate and the second coordinate, and divide the target medical image into the first target knee image and the second target knee image based on the reference coordinate (or reference line).

In this case, the reference coordinate may be obtained by assigning a preset weight to the first coordinate and the second coordinate. For example, the first feature region related to the first knee and the second feature region related to the second knee may anatomically correspond to each other or may be the same region. In this case, the medical image analysis apparatus 1000 may acquire the average value of the first coordinate corresponding to the first feature region and the second coordinate corresponding to the second feature region as the reference coordinate. As another example, the first feature region (e.g., a region inside the knee) related to the first knee and the second feature region (e.g., a region outside the knee) related to the second knee may be anatomically different regions. In this case, the medical image analysis apparatus 1000 may acquire the reference coordinate by assigning a first weight to the first coordinate corresponding to the first feature region and a second weight having a relatively smaller value than the first weight to a second coordinate corresponding to the second feature region. However, this is only an example, and it may be implemented to acquire the first target knee image and the second target knee image from the target medical image according to any appropriate standard based on the first coordinate and the second coordinate.

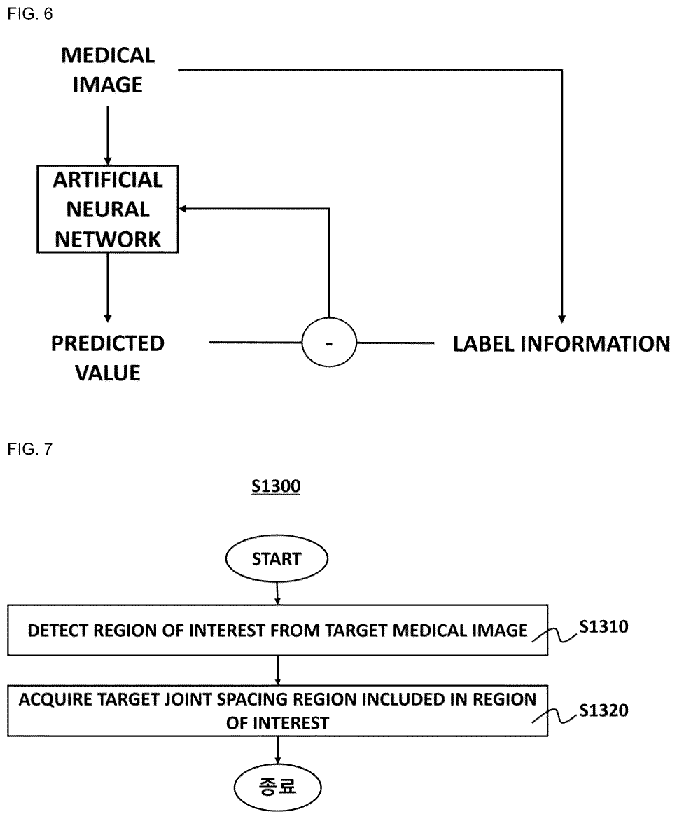

Referring to FIG. 6, FIG. 6 is a diagram illustrating a method of training a knee detection model according to an embodiment of the present application.

The neural network model may include an input layer, an output layer, and a hidden layer. The input layer may receive a medical image, and the output layer may output a predicted value (e.g., coordinate information of the feature region) for the feature region included in the medical image. The hidden layer may have a plurality of nodes connecting between the input layer and the output layer.

The medical image analysis apparatus 1000 according to an embodiment of the present application may train an artificial neural network to output the predicted value for the feature region based on the medical image. Specifically, the medical image analysis apparatus 1000 may input the medical image to the input layer, and acquire the predicted value for the feature region through the output layer. In addition, the medical image analysis apparatus 1000 may adjust weights (or parameters) of nodes included in the hidden layer based on a difference between label information (e.g., the label information includes a value defining a region of at least one of right femur medial, right fibula, left femur medial, left fibula, right letter, and left letter) and the predicted value. Specifically, the medical image analysis apparatus 1000 may train the neural network model by repeatedly adjusting the weights (or parameters) of the nodes included in the hidden layer so that the difference between the label information related to the feature region and the predicted value is minimized. Therefore, the trained neural network model (i.e., knee detection model) may output information on the feature region included in the medical image based on the medical image.

To quantify the joint spacing of the knee or analyze arthritis, it is important to acquire the medical image related to the knee to be analyzed. When the medical image is captured to diagnose knee-related diseases, the medical image including both the left and right knee regions are acquired. In this case, when the medical image including both the left and right knee regions is input to the neural network model for medical image analysis, which will be described below, an error may exist in the analysis results acquired through the neural network model. In addition, conventionally, the entire medical image is randomly divided into ½ points on a horizontal axis, and the left and right knee regions included in the entire medical image are divided into the image including the left knee region and the image including the right knee region. However, according to the related art, in the case of an image in which the knee is biased to the left or right or an image in which both knees are narrowly positioned, the knee region is partially cut off, and there have been cases where the medical image analysis could not be performed.

According to the method of acquiring a target knee image according to an embodiment of the present application, the first feature region related to the left knee and the second feature region related to the right knee may be acquired through the knee detection model, and the target medical image may be divided into the first target knee image related to the left knee and the second target knee image related to the right knee based on the first feature region and the second feature region, thereby reducing the errors in the medical image analysis, which will be described below, and increasing the accuracy of the medical image analysis.

Hereinafter, the detection of the ROI or the target joint spacing region from the target medical image will be described. However, this is for convenience of description, and the target medical image may be used with a meaning including the first target knee image and/or the second target knee image described above.

Referring back to FIG. 3, in the detecting of the target joint spacing region (S1300), the medical image analysis apparatus 1000 may detect the target joint spacing region from the target medical image. As an example, the medical image analysis apparatus 1000 may be implemented to acquire the region including the joint part included in the target medical image as the ROI from the target medical image, and acquire the target joint spacing region by precisely analyzing the ROI. As another example, the medical image analysis apparatus 1000 may be implemented to acquire the target joint spacing region included in the target medical image from the target medical image.

Hereinafter, the description will focus on the embodiment in which the ROI is acquired from the target medical image and the target joint spacing region is acquired by precisely analyzing the ROI. However, this is only an example and should not be interpreted as limiting.

Referring to FIGS. 7 and 8, FIG. 7 is a flowchart embodying an operation of detecting a target joint spacing region according to an embodiment of the present application. FIG. 8 is a diagram illustrating an aspect of detecting an ROI and a target joint spacing region according to an embodiment of the present application.

The acquiring of the target joint spacing region (S1300) may include detecting the ROI from the target medical image (S1310) and acquiring the target joint spacing region included in the ROI (S1320).

In the detecting of the ROI from the target medical image (S1310), the medical image analysis apparatus 1000 may be implemented to detect the region including the joint part as the ROI from the target medical image. For example, the medical image analysis apparatus 1000 may acquire the region including the joint part as the ROI by using any appropriate artificial intelligence technique. Specifically, the medical image analysis apparatus 1000 may be implemented to receive the target medical image and detect an ROI using a first neural network model trained to output the region including the joint part as the ROI.

Here, the first neural network model may be trained based on the medical image and the label information allocating the ROI to the medical image. In this case, the label information may be automatically allocated to the medical image using any software or may be manually allocated to the medical image by any operator. Meanwhile, the label information may include a label related to the ROI related to one or more joints. Specifically, the label information may include a first label related to the knee region (e.g., the knee region may be assigned to the knee region centered on the joint) and/or a second label (e.g., a region related to medial femur, femur lateral, tibia medial, tibia lateral, etc.) related to a main occurrence region of osteophyte. However, this is only an example, and the label information may further include a label related to any appropriate region, including a third label related to the cartilage region.

More specifically, the first neural network model may be trained to receive the medical image and to minimize a difference between an output value and the label information related to the joint part region. Meanwhile, the first neural network model may be a deep learning-based artificial neural network model. Specific examples of the artificial neural network may include a convolutional neural network, a recurrent neural network, a deep neural network, a generative adversarial network, and the like. However, this is merely an example and should be interpreted in a comprehensive sense including all of the above-described artificial neural networks, various other types of artificial neural networks, and artificial neural networks of a combination thereof, and is not necessarily the deep learning-based artificial neural network model.

In the detecting of the target joint spacing region included in the ROI (S1300), the medical image analysis apparatus 1000 may detect the target joint spacing region from the target medical image. Specifically, the medical image analysis apparatus 1000 may perform segmentation on the ROI of the target medical image to acquire the target joint spacing region including the inter-joint region. For example, the segmentation of the ROI may be performed using any appropriate artificial intelligence technique.

Referring back to FIG. 8, the medical image analysis apparatus 1000 may be implemented to detect the target joint spacing region by using a second neural network model trained to receive the target medical image including the ROI and output the inter-joint region.

According to an example, the neural network model may be used as a model for acquiring the target joint spacing region. The neural network model may be provided as a machine learning model. As a representative example of the machine learning model, there may be an artificial neural network. Specifically, a representative example of the artificial neural network is a deep learning-based artificial neural network that includes an input layer that receives data, an output layer that outputs a result, and a hidden layer that processes data between the input and output layers. Specific examples of the artificial neural network include a convolution neural network, a recurrent neural network, a deep neural network, a generative adversarial network, and the like. In the present specification, the neural network should be interpreted in a comprehensive sense including all of the artificial neural networks described above, various other types of artificial neural networks, and artificial neural networks in a combination thereof, and does not necessarily have to be a deep learning series.

In addition, the machine learning model does not necessarily have to be in the form of the artificial neural network model, and in addition, there may be nearest neighbor algorithm (KNN), random forest, support vector machine (SVM), principal component analysis (PCA), etc. Alternatively, the above-described techniques may include an ensemble form or a form in which various other methods are combined. On the other hand, it is stated in advance that the artificial neural network can be replaced with another machine learning model unless otherwise specified in the embodiments mainly described with the artificial neural network.

Furthermore, in the present specification, an algorithm for acquiring the target joint spacing region is not necessarily limited to the machine learning model. That is, the algorithm for acquiring the target joint spacing region may include various judgment/determination algorithms instead of the machine learning model. Therefore, in this specification, it is to be understood that the algorithm for acquiring the target joint spacing region should be understood with a comprehensive meaning including all types of algorithms for calculating the joint spacing region based on the medical image. Hereinafter, however, for convenience of description, the artificial neural network model will be mainly described.

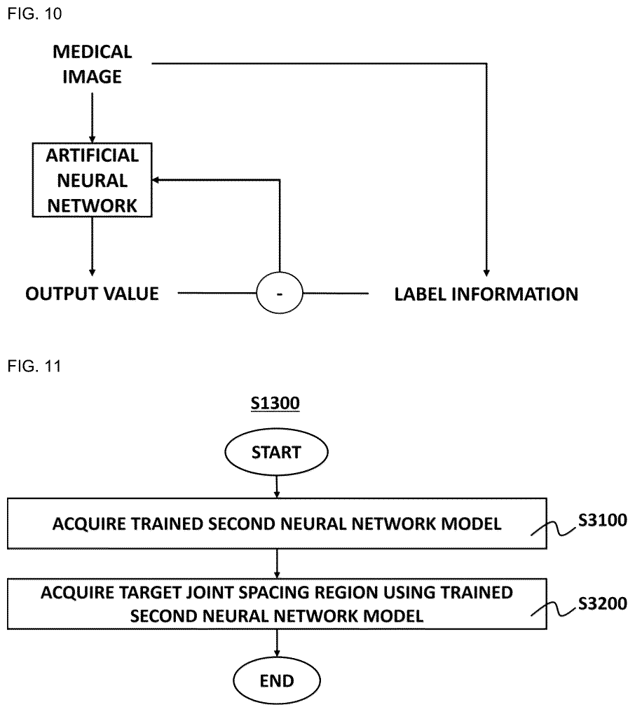

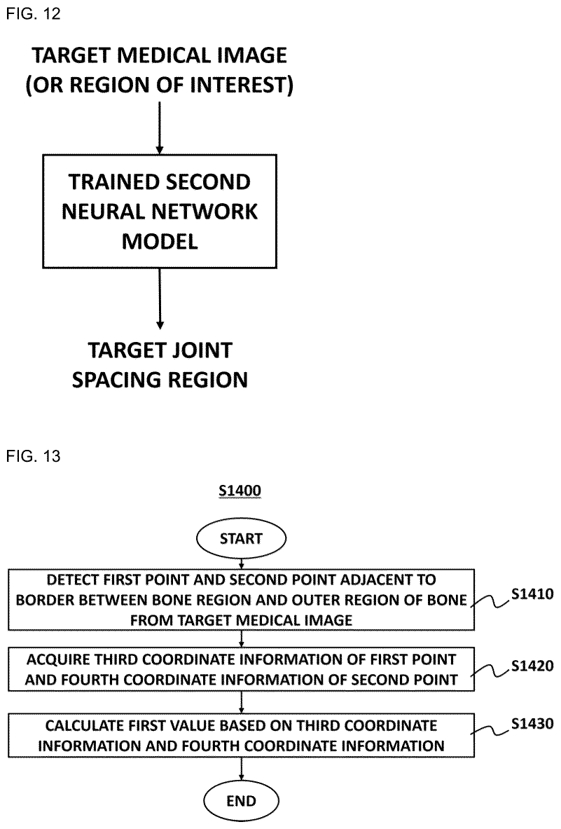

Hereinafter, the content of acquiring the target joint spacing region using the neural network model according to the embodiment of the present application will be described with reference to FIGS. 9 to 12. Specifically, the content of training the neural network model for acquiring the target joint spacing region according to the embodiment of the present application will be described with reference to FIGS. 9 and 10. In addition, the content of acquiring the target joint spacing region using the trained neural network model with reference to FIGS. 11 and 12 will be described.

Referring to FIG. 9, FIG. 9 is a flowchart illustrating a method of training a neural network model for acquiring a target joint spacing region according to an embodiment of the present application. A method of training a neural network model for acquiring a target joint spacing region may be performed in the medical image analysis apparatus 1000. However, the method of training a neural network model for acquiring a target joint spacing region may also be performed in a separate external device from the medical image analysis apparatus 1000. Hereinafter, it will be described that the training of the neural network model for acquiring the target joint spacing region in the medical image analysis apparatus 1000 is performed. However, this is only an example, and should not be construed as limiting.

The method of training a neural network model for acquiring a target joint spacing region according to the embodiment of the present application may include acquiring a medical image database (S2100), preparing a training set (S2200), training a neural network (S2300), and acquiring the trained second neural network model (S2400).

In the acquiring of the medical image database (S2100), the medical image analysis apparatus 1000 may acquire a medical image database including a plurality of medical images from the medical image acquisition apparatus 100 or an external device including any database.

In the preparing of the training set (S2200), the medical image analysis apparatus 1000 may acquire the prepared training set by allocating label information to the inter-joint region included in the medical image. The operation of allocating the label information to the inter-joint region may be performed using any appropriate software, or may be manually performed by any operator, similar to the above.

In the training of the neural network (S2300), the medical image analysis apparatus 1000 may train the neural network model based on the medical image and the training set.

Referring to FIG. 10, FIG. 10 is a flowchart illustrating an aspect of training a neural network model for acquiring a target joint spacing region according to an embodiment of the present application.

The neural network model may include an input layer, an output layer, and a hidden layer. The input layer may receive a medical image, and the output layer may output an output value related to the inter-joint region. The hidden layer may have a plurality of nodes connecting between the input layer and the output layer.

The medical image analysis apparatus 1000 according to the embodiment of the present application may train a neural network to output joint spacing region information indicating an inter-joint region based on a medical image. Specifically, the medical image analysis apparatus 1000 may input the medical image to the input layer, and may acquire the output value related to the inter-joint region through the output layer. In addition, the medical image analysis apparatus 1000 may adjust weights (or parameters) of nodes included in the hidden layer based on the difference between the label information and the output value related to the joint spacing region included in the training set. For example, the medical image analysis apparatus 1000 may input a first medical image acquired from the medical image database to the input layer, and update the weights (or parameters) of the nodes included in the hidden layer based on the difference between the output value output through the output layer and the first label information allocated to the joint spacing region of the first medical image. In addition, the medical image analysis apparatus 1000 may input an Nth medical image acquired from the medical image database to the input layer, and repeatedly update the weights (or parameters) of the nodes included in the hidden layer based on the difference between the output value output through the output layer and the Nth label information allocated to the joint spacing region of the Nth medical image.

Specifically, the medical image analysis apparatus 1000 may train the neural network model by repeatedly adjusting the weights (or parameters) of the nodes included in the hidden layer so that the difference between the label information and the output value related to the joint spacing region is minimized.

Referring back to FIG. 9, the method of training a neural network model for acquiring a target joint spacing region according to the embodiment of the present application may include acquiring a trained second neural network model (S2400). In the acquiring of the trained second neural network model (S2400), the medical image analysis apparatus 1000 may acquire the output value output through the output layer and the weights or parameters of the nodes included in the hidden layer that is trained so that the output value and label information output through the output layer are minimized. Alternatively, in the acquiring of the trained second neural network model (S2400), the medical image analysis apparatus 1000 may acquire the second neural network model that includes the hidden layer including the nodes having the above-described weights or parameters.

Meanwhile, although not illustrated in FIG. 9, the neural network model for acquiring the target joint spacing region according to the embodiment of the present application may further include verifying the neural network. For example, the medical image analysis apparatus 1000 may verify the neural network model based on at least a part of the training set. Specifically, the medical image analysis apparatus 1000 may input at least some of the medical images included in the training set to the input layer of the neural network model, and acquire the output value output through the output layer. In addition, the medical image analysis apparatus 1000 may compare similarity between the output value and the label information related to the medical image included in the training set to verify whether the weights (or parameters) of the node included in the hidden layer of the neural network model are appropriate.

Meanwhile, the target medical image may include a plurality of joint regions. For example, a bone image may include several joint regions. In this case, the medical image analysis apparatus 1000 according to the embodiment of the present application may be implemented to train the neural network model for each joint region and detect the joint spacing region using at least one neural network model trained for each joint region. For example, the medical image analysis apparatus 1000 may be configured so that, for the first joint region, the joint spacing region included in the first joint region is detected using the trained first joint detection neural network model, and for the second joint region, the joint spacing region included in the second joint region is detected using the trained second joint detection neural network model. However, this is only an example, and a single neural network model may be used in a case where a shape of the inter-joint region is similar.

Referring to FIG. 11, FIG. 11 is a flowchart embodying an operation of detecting a target joint spacing region according to an embodiment of the present application.

The detecting of the target joint spacing region (S1300) according to the embodiment of the present application may include acquiring the trained second neural network model (S3100) and acquiring the target joint spacing region using the trained second neural network model (S3200).

In the acquiring of the trained second neural network model (S3100), the medical image analysis apparatus 1000 may acquire the trained second neural network model. For example, the weights or parameters of the nodes of the second neural network model may be acquired. As another example, the medical image analysis apparatus 1000 may acquire the neural network that includes the hidden layer including the nodes having the weights or parameters acquired through the training.

In the acquiring of the target joint spacing region using the trained second neural network model (S3200), the medical image analysis apparatus 1000 according to the embodiment of the present application may acquire the target joint spacing region based on the trained second neural network model and the target medical image.

Refer to FIG. 12. FIG. 12 is a schematic diagram illustrating an aspect of acquiring a target joint spacing region using a trained neural network model according to an embodiment of the present application.

The medical image analysis apparatus 1000 may input the target medical image (or a target medical image including an ROI) to the input layer of the trained second neural network model, and acquire the information related to the target joint spacing region through the output layer. Since the trained second neural network model is trained to output the joint spacing region information based on the medical image, the medical image analysis apparatus 1000 may acquire the target joint spacing region included in the target medical image (or a medical image including an ROI) based on the trained second neural network model and the target medical image.

Referring back to FIG. 3, the medical image analysis method according to the embodiment of the present application may include acquiring the first value related to the width of the joint part (S1400).

Referring to FIG. 13, FIG. 13 is a flowchart embodying an operation (S1400) of acquiring a first value related to a width of a joint part according to an embodiment of the present application.

The acquiring of the first value (S1400) according to an embodiment of the present application may include detecting a first point and a second point adjacent to a border between a bone region and an outer region of a bone from the target medical image (S1410), acquiring third coordinate information of the first point and fourth coordinate information of the second point (S1420), and calculating the first value based on the third coordinate information and the fourth coordinate information (S1430).

In the detecting of the first point and the second point adjacent to the border between the bone region and the outer region of the bone from the target joint spacing region (S1410), the medical image analysis apparatus 1000 may detect the inter-joint region and the adjacent bone region and the outer region of the bone included in the target medical image. In detail, the medical image analysis apparatus 1000 may detect a first point P1 and a second point P2 adjacent to a border between a bone region R1 adjacent to a joint and an outer region R2 of a bone from the target medical image (or the medical image including the ROI).

For example, the medical image analysis apparatus 1000 may detect the first point P1 and the second point P2 using any image processing technique. For example, the medical image analysis apparatus 1000 may process the target medical image by using any image processing technique, and detect the first point P1 and the second point P2 based on the brightness of the processed target medical image. Specifically, the medical image analysis apparatus 1000 may acquire the border between the bone region R1 and the outer region R2 of the bone based on a difference between first brightness of the bone region R1 and second brightness of the outer region R2 of the bone, and acquire the first point P1 and the second point P2 adjacent to the border. Specifically, the medical image analysis apparatus 1000 may acquire the tibia region of the medical image based on the difference between the first brightness and the second brightness, and acquire the first point P1 and the second point located on both outer sides of the tibia region.

As another example, the medical image analysis apparatus 1000 may detect the first point P1 and the second point P2 using any image processing technique. For example, the medical image analysis apparatus 1000 may train a neural network model for acquiring both end points of a bone based on the medical image and label information on both end points of the bone adjacent to the inter-joint region. In this case, the medical image analysis apparatus 1000 may detect both end points of the bone, for example, the first point P1 and the second point P2, using the trained neural network model. Specifically, the medical image analysis apparatus 1000 may detect the first point P1 and the second point P2 through the neural network model trained based on the medical image and the label information on the tibia region of the medical image.

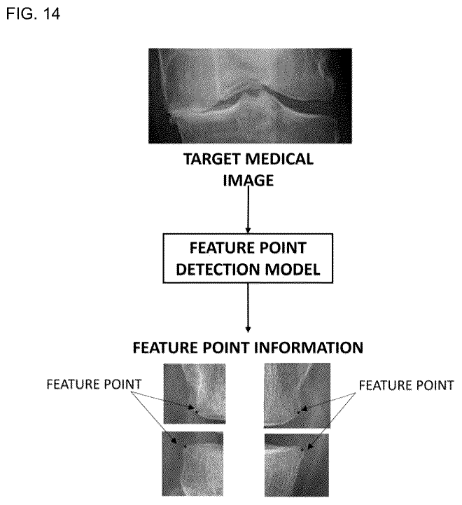

FIG. 14 is a diagram illustrating an aspect of detecting the first point P1 and the second point P2 according to an embodiment of the present application.

The osteophyte is a bone spur generated by degenerative reactions, and errors may occur when calculating the width of the joint part based on the osteophyte region included in the target medical image. Therefore, it is important to detect the tibia region (e.g., the region adjacent to the border of the osteophyte region and the tibia region) adjacent to the region where the osteophyte starts.

The medical image analysis apparatus 1000 according to an embodiment of the present application may detect the feature point information (e.g., feature point coordinates) of the tibia region and/or femur region using the feature point detection model trained based on the training set including the medical image (e.g., the image including the main occurrence region of the osteophyte) and the label information allocating the feature point of the tibia region and/or the feature point of the femur region included in the medical image. Specifically, the medical image analysis apparatus 1000 may detect the feature points of the tibia region or femur region from the target medical image (e.g., a medical image including an ROI related to osteophyte) through the feature point detection model, and acquire the feature points of the tibia region as the first point P1 and the second point P2. Meanwhile, the feature points of the femur region may be used to acquire the section of interest of the joint spacing region, which will be described later.

Meanwhile, the feature point detection model may be trained through deep high-resolution representation learning (HRNet), which shows excellent performance in pose estimation.

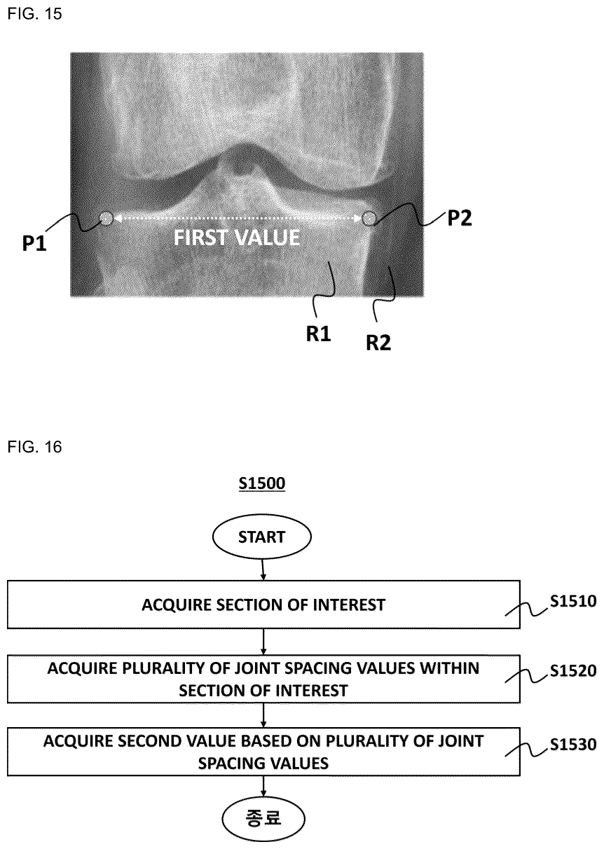

FIG. 15 is a diagram illustrating an aspect of acquiring a first value according to an embodiment of the present application.

In the obtaining of the third coordinate information of the first point and the fourth coordinate information of the second point (S1420), the medical image analysis apparatus 1000 may acquire the coordinate information of the detected first point P1 and second point P2. Specifically, the target medical image may include a plurality of cells (for example, pixels or voxels), and the target medical image may include the coordinate information of the cells. In this case, the medical image analysis apparatus 1000 may acquire the third coordinate information based on the coordinates of the cell corresponding to the first point P1, and acquire the fourth coordinate information based on the coordinate of the cell corresponding to the second point P2.

In the calculating of the first value based on the third coordinate information and the fourth coordinate information (S1430), the medical image analysis apparatus 1000 may acquire the first value related to the width of the joint part based on the third coordinate information and the fourth coordinate information. For example, as illustrated in FIG. 11, in the target medical image in which two bones are positioned vertically, when the third coordinate information of the first point P1 (for example, e1(x,y)) and the fourth coordinate information of the second point P2 (e.g., e2(x,y)), which are both end points of the bone, are acquired, the medical image analysis apparatus 1000 may be implemented to calculate the first value related to the width of the joint part as |e1(x,y)−e2(x,y) | using a method of calculating a Euclidean distance. However, this is only an example of description, and the medical image analysis apparatus 1000 may be implemented to calculate the first value related to the width of the joint part in consideration of the alignment direction of the target medical image.

Referring back to FIG. 3, the medical image analysis method according to the embodiment of the present application may include acquiring the second value related to the joint spacing (S1500).

Referring to FIGS. 16 and 17, FIG. 16 is a flowchart embodying an operation of acquiring a second value related to the joint spacing (S1500) according to an embodiment of the present application. FIG. 17 is a diagram illustrating an aspect of acquiring a second value according to an embodiment of the present application.

The acquiring of the second value related to the joint spacing (S1500) according to the embodiment of the present invention may include acquiring a section of interest (S1510), acquiring a plurality of joint spacing values within the section of interest (S1520), and acquiring the second value based on the plurality of joint spacing values (S1530).

In the acquiring of the section of interest from the joint spacing region (S1510), the medical image analysis apparatus 1000 may acquire the section of interest. As an example, the medical image analysis apparatus 1000 may acquire the inter-joint region known to have high clinical importance in identifying the joint condition as a section of interest. For example, in the case of a knee joint, a section that has cartilage and is known as a region related to severe pain due to arthritis may be acquired as a section of interest.

For example, the section of interest may be automatically acquired using both end points (for example, the first point P1 and the second point P2) of the bone described above, the width (for example, the first value) between both end points of the bone, the statistical ratio of the clinically important section described above, or the like. As another example, a user may input the section of interest through any input unit, and the medical image analysis apparatus 1000 may acquire the section of interest based on the user input.

On the other hand, the section of interest may be divided into several subsections, and may be continuous or discontinuous.

In the acquiring of the plurality of joint spacing values within the section of interest (S1520), the medical image analysis apparatus 1000 may acquire at least one joint spacing value within the section of interest. For example, the medical image analysis apparatus 1000 may acquire at least one joint spacing value of the inter-joint region within the first section of interest. In addition, the medical image analysis apparatus 1000 may acquire at least one joint spacing value of the joint spacing region within the second section of interest.

The coordinate information of the joint spacing region may be used for the medical image analysis apparatus 1000 to acquire the joint spacing value. For example, the medical image analysis apparatus 1000 may be implemented to acquire the joint spacing value based on the coordinate information of the boundary defining the joint spacing region within the section of interest (for example, a first section of interest or a second section of interest). However, this is only an example, and the medical image analysis apparatus 1000 may acquire the joint spacing value within the section of interest using any appropriate method.

In the acquiring of the second value based on the plurality of joint spacing values (S1530), the medical image analysis apparatus 1000 may acquire the second value related to the joint spacing based on at least one joint spacing value acquired from the target joint spacing region within the section of interest. For example, the medical image analysis apparatus 1000 may acquire a minimum value among the plurality of joint spacing values as the second value related to the joint spacing. As another example, the medical image analysis apparatus 1000 may acquire an average value of the plurality of joint spacing values as the second value related to the joint spacing. As another example, the medical image analysis apparatus 1000 may acquire the second value by assigning any appropriate weight to the minimum value among the plurality of joint spacing values and the average value of the plurality of joint spacing values.

Referring back to FIG. 3, an operation (S1600) of calculating the target joint condition indicator indicating the joint condition according to an embodiment of the present application may be included.

In the calculating of the target joint condition indicator indicating the joint condition (S1500), the medical image analysis apparatus 1000 may calculate the target joint condition indicator based on the first value related to the width of the joint part and the second value related to the joint spacing. For example, the medical image analysis apparatus 1000 may calculate the target joint condition indicator defined as a ratio of the second value to the first value. Specifically, when the second value is the minimum joint spacing value, the medical image analysis apparatus 1000 may calculate the minimum joint spacing value compared to the first value (the width value of the joint part) as the target joint condition indicator. Alternatively, when the second value is the average space value, the medical image analysis apparatus 1000 may calculate the average joint spacing value compared to the first value (the width value of the joint part) as the target joint condition indicator.

The target joint condition indicator acquired according to the embodiment of the present application may be quantified by linking the joint spacing value that is affected by various external factors such as race, gender, and age with the width of the joint part. Accordingly, the target joint condition indicator acquired according to the embodiment of the present application may minimize the influence of various external factors such as race, gender, and age, and provide objective joint condition information.

Meanwhile, the medical image analysis apparatus 1000 according to the embodiment of the present application may perform an operation of comparing and analyzing a target joint condition indicator with a reference joint condition indicator of a normal joint group. Specifically, the medical image analysis apparatus 1000 may acquire a joint condition data set from any database. In this case, the joint condition data set may include a reference medical image representing a normal joint, a width value of a joint part calculated from the reference medical image, a joint spacing value calculated from the reference medical image, and the like.

The medical image analysis apparatus 1000 may calculate a reference joint condition indicator of a normal group from the joint condition data set. For example, the medical image analysis apparatus 1000 may calculate the first joint condition indicator (for example, a minimum joint spacing value compared to a width value of a joint part) from a first reference medical image related to the normal joint, and the second condition indicator (for example, the minimum joint spacing value compared to the width value of the joint part) from the medical image related to the normal joint. In addition, the medical image analysis apparatus 1000 may calculate the reference joint condition indicator based on the first joint condition indicator and the second joint condition indicator. For example, the reference joint condition indicator may be calculated as the average value of the plurality of joint condition indicators of the normal group, including the first joint condition indicator and the second joint condition indicator.

The medical image analysis apparatus 1000 according to the embodiment of the present application may quantify the target joint condition indicator by comparing the target joint condition indicator with the reference joint condition indicator. As an example, the medical image analysis apparatus 1000 may calculate a reduction rate of the target joint condition indicator compared to the reference joint condition indicator. As an example, the reduction rate may be defined as ((reference joint condition indicator−target joint condition indicator)/reference joint condition indicator)*100. For example, when a reference joint condition indicator (for example, a minimum joint spacing value compared to the width value of the joint part), which is an average value of joint condition indicators of normal groups, is 0.22, and the target joint condition indicator (for example, the minimum joint spacing value compared to the value of the joint width part) is 0.055, the reduction rate may be calculated as about 75%. In other words, the medical image analysis apparatus 1000 may quantify the reduction rate indicating a state in which a joint condition of a subject to be analyzed has a reduction rate of 75% compared to the normal joint, and thus the joint spacing is narrowed compared to the normal joint.

As another example, when a reference joint condition indicator (for example, the minimum joint spacing value compared to the width value of the joint part), which is an average value of joint condition indicators of knees of normal groups, is 0.06, and the target joint condition indicator (for example, the minimum joint spacing value compared to the value of the joint width part) is 0.015, the reduction rate may be calculated as about 75%. In other words, the medical image analysis apparatus 1000 may quantify the reduction rate indicating a state in which the joint condition of the subject to be analyzed has a reduction rate of 75% compared to a normal knee, and thus the knee joint spacing of the subject to be analyzed is narrowed compared to the normal knee joint. However, the above-described numerical values are merely examples for convenience of description, and should not be construed as limiting.

Through the above-described comparative analysis, the medical image analysis apparatus 1000 according to the embodiment of the present application may compare states between joints of the same person or analyze the change in the joint conditions over time. In addition, the medical image analysis apparatus 1000 may provide information on a joint condition of a subject to be analyzed by quantifying a joint condition between other persons.

The medical image analysis apparatus 1000 according to an embodiment of the present application may be implemented to analyze the osteophyte region and/or the above-described joint spacing region, which is detected from the above-described target medical image and classify osteoarthritis severity levels based on the analysis results. Specifically, the medical image analysis apparatus 1000 may automatically classify an arthritis severity grade from the osteophyte region and/or joint spacing region of the target medical image using the artificial intelligence model trained based on the training set composed of the medical images and standard arthritis severity grade information (e.g., grades according to Kellgren Lawrence classification) allocated to each medical image.

In addition, the medical image analysis apparatus 1000 according to an embodiment of the present application may be configured to classify bone proliferation grades based on the osteophyte region. It is known that bone proliferation depends on the severity of degenerative arthritis, and that the degree of bone proliferation increases as the severity increases. The medical image analysis apparatus 1000 may automatically classify the bone proliferation grade from the osteophyte region of the target medical image using the artificial intelligence model trained based on the training set composed of the medical images and bone proliferation grade information allocated to each medical image.

In addition, the medical image analysis apparatus 1000 according to an embodiment of the present application may integrally analyze the joint condition based on the target joint condition indicator and/or the osteophyte region analysis results. For example, according to the Kellgren-Lawrence classification, when the joint spacing narrowing is 50% or more and secondary findings of osteophyte, sclerosis, and defects are also detected, the joint condition may be determined as degenerative arthritis grade 3. In addition, according to the Kellgren-Lawrence classification, when the joint spacing narrowing is 75% or more and secondary findings of osteophyte, sclerosis, and defects are also detected, the joint condition may be determined as degenerative arthritis grade 4.

The medical image analysis apparatus 1000 according to an embodiment of the present application may calculate the integrated analysis results related to degenerative arthritis based on the result of comparing the target joint condition indicator and the preset joint spacing reduction rate condition and the analysis result of the osteophyte region. For example, when the target joint condition indicator is calculated as 60%, the medical image analysis apparatus 1000 may compare the target joint condition indicator and the preset joint spacing reduction rate condition to determine that the target joint condition indicator corresponds to a first section (e.g., 50% or more and less than 75%). In addition, the medical image analysis apparatus 1000 may calculate the integrated analysis results indicating that the target joint condition corresponds to the degenerative arthritis grade 3 based on the determination result that the target joint condition indicator corresponds to the first section and the determination that the osteophyte region is detected.

The medical image analysis apparatus 1000 according to an embodiment of the present application may perform an operation of visualizing any analysis results of the target medical image.

FIG. 18 is a diagram illustrating an example of visualization information of an analysis result of the medical image analysis apparatus 1000 according to an embodiment of the present application. Specifically, the medical image analysis apparatus 1000 may visually process the analysis region of the target medical image, which is the determination standard of the degenerative severity grade and osteophyte severity grade. In this case, the medical image analysis apparatus 1000 may display different colors in corresponding regions according to the severity grade and provide the displayed colors to the user. In addition, the medical image analysis apparatus 1000 may display a region (i.e., the area in which the joint spacing is the narrowest), in which the target joint condition indicator is the smallest, in the section of interest of the joint spacing region in the target medical image and provide the region to the user.

Meanwhile, although not illustrated, the medical image analysis apparatus 1000 according to the embodiment of the present application may output any analysis results through any output unit, including the joint condition information or the comparative analysis result with a normal group. Alternatively, the medical image analysis apparatus 1000 according to an embodiment of the present application may transmit the analysis results to any external device including the medical image acquisition device 100. Any external device that receives the analysis results may output the analysis results for the target medical image to the user through any output unit.

According to the medical image analysis method, the medical image analysis apparatus, and the medical image analysis system according to an embodiment of the present application, it is possible to provide objective joint condition information by minimizing the influence of external factors such as a body type, a race, and a gender. In addition, according to the medical image analysis method, apparatus and system according to the embodiment of the present application, it is possible to more accurately estimate the joint condition based on the objective joint condition information.

Various operations of the medical image analysis apparatus 1000 described above may be stored in the memory 1200 of the medical image analysis apparatus 1000, and the controller 1300 of the medical image analysis apparatus 1000 may be provided to perform the operations stored in the memory 1200.

Features, structures, effects, etc., described in the above embodiments are included in at least an embodiment of the present disclosure, and are not necessarily limited only to an embodiment. Furthermore, features, structures, effects, etc., illustrated in each embodiment can be practiced by being combined with other embodiments or modified by those of ordinary skill in the art to which the embodiments pertain. Accordingly, content related to such combinations and modifications is to be interpreted as falling within the scope of the present disclosure.

Although exemplary embodiments have been mainly described hereinabove, these are merely exemplary and do not limit the present disclosure. Those skilled in the art to which the present disclosure pertains may understand that several modifications and applications that are not described in the present specification may be made without departing from the spirit of the present disclosure. That is, each component specifically shown in the embodiment may be implemented by modification. In addition, differences associated with these modifications and applications are to be interpreted as falling within the scope of the present invention as defined by the following claims.

1. A medical image analysis method, which is performed by an apparatus for acquiring a medical image and performing a morphological analysis of a joint based on the medical image, the medical image analysis method comprising:
acquiring a target medical image;
acquiring a trained knee detection model;
acquiring a first feature region related to a first knee from the target medical image through the knee detection model;
acquiring a second feature region related to a second knee from the target medical image through the knee detection model; and
acquiring a first target knee image related to the first knee and a second target knee image related to the second knee based on the first feature region and the second feature region of the target medical image.

2. The medical image analysis method of claim 1, wherein the acquiring of the first target knee image related to the first knee and the second target knee image related to the second knee includes:
acquiring a first coordinate related to the first feature region;
acquiring a second coordinate related to the second feature region;
acquiring a reference coordinate by assigning a preset weight to each of the first and second coordinates; and
dividing the target medical image into the first target knee image and the second target knee image based on the reference coordinate.

3. The medical image analysis method of claim 1, wherein the first feature region is a region of at least one of right femur medial, right fibula, left femur medial, left fibula, right letter, and left letter, and
the second feature region is a region of at least one of the right femur medial, the right fibula, the left femur medial, the left fibula, the right letter, and the left letter.

4. The medical image analysis method of claim 1, wherein the knee detection model is configured to receive the target medical image and detect the first feature region or the second feature region included in the target medical image.

5. The medical image analysis method of claim 1, wherein the knee detection model includes an input layer that receives the medical image, an output layer that outputs a predicted value, and a hidden layer that connects the input layer and the output layer,
is trained based on a training set composed of the medical image and label information allocated to the feature region of the medical image, and
is trained by adjusting a parameter of a node included in the hidden layer based on a difference between the predicted value output through the output layer and the label information.

6. The medical image analysis method of claim 5, wherein the label information includes a value defining at least one of right femur medial, right fibula, left femur medial, left fibula, right letter, and left letter.

7. A computer-readable recording medium on which a program for executing the method of claim 1 is recorded.

8. A medical image analysis apparatus for acquiring a medical image and acquiring a knee image to be analyzed from the medical image, the medical image analysis apparatus comprising:
an image acquisition unit that acquires a target medical image; and
a controller that acquires a target knee image to be analyzed based on the target medical image,
wherein the controller is configured to acquire the target medical image, acquire a trained knee detection model, acquire a first feature region related to a first knee from the target medical image through the knee detection model, acquire a second feature region related to a second knee from the medical image through the knee detection model, and acquire a first target knee image related to the first knee and a second target knee image related to the second knee based on the first feature region and the second feature region of the target medical image.
